(19) United States
(12) Patent Application Publication
(10) Pub. No.: US 2006/0292639 A1
(43) Pub. Date: Dec. 28, 2006

(54) Splice variant of the vanilloid receptor VR1A

(75) Inventors: Per-Erik Mansson, West Chester, PA (US); Diane L. DeHaven-Hudkins, Chester Springs, PA (US); Lily Lu, Mickleton, NJ (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Adolor Corporation, Exton, PA (US)

(21) Appl. No.: 11/201,937

(22) Filed: Aug. 11, 2005

Related U.S. Application Data
(60) Provisional application No. 60/601,555, filed on Aug. 13, 2004.

(51) Int. Cl. G01N 33/567; C07H 21/04; C12P 21/06; C07K 14/705; G01N 33/53

(52) U.S. Cl. 435/7.2; 435/69.1; 435/320.1; 435/325; 530/350; 536/23.5; 530/388.22

(57) ABSTRACT

The invention provides a splice variant of the vanilloid receptor termed “VR1A.” VR1A has a unique N-terminus as compared with wild type VR1. The invention provides nucleic acid sequences encoding VR1A, expression vectors for expressing VR1A, amino acid sequences of VR1A, antibodies against VR1A and methods of making and using the polynucleotides, polypeptides and antibodies of the invention.

### CROSS-REFERENCE TO RELATED APPLICATIONS

This application claims benefit of U.S. Provisional Application No. 60/601,555 filed Aug. 13, 2004, the disclosure of which is hereby incorporated by reference herein in its entirety.

### FIELD OF THE INVENTION

The invention relates to nucleic acid and amino acid sequences of a human vanilloid receptor splice variant and to the use of these sequences.

### BACKGROUND OF THE INVENTION

Vanilloid receptor 1 (VR1) encodes a ligand-gated, cationic channel (Oh et al., 1996) that is activated by capsaicin, the potent pain-causing principal of hot chili peppers (Caterina et al., 1997). The VR1 receptor belongs to TRPV, a subset of the TRP (transient receptor potential) channel family, and members of TRPV channels are typified by having six transmembrane domains (TM), cytosolic N- and C-terminal ends, ankyrin repeats at the N-terminus, and a re-entry loop between TM5 and TM6 (Clapham, 2003). VR1/TRPV1 has been cloned from both rat (Caterina et al., 1997) and human (Hayes et al., 2000) dorsal root ganglia (DRG) and the amino acid sequences revealed an 86% homology, suggesting a high degree of species conservation.

As a molecular target, the VR1 receptor is capable of responding to multiple stimuli like heat, acid and the hot ingredient in chili pepper, capsaicin, suggesting a unique role in the response to painful stimuli. In response to heat, low pH and capsaicin, calcium flows from the extracellular lumen through the channel to initiate a cascade of events, which leads to the sensation of pain (Caterina et al., 1997; Szallasi and Blumberg, 1999; Caterina and Julius, 2001; Julius and Basbaum, 2001). The putative endogenous ligand, 12-hydroperoxytetraenoic acid, a product of 12-lipoxygenase, activates and binds to VR1, and shares structural similarities with capsaicin (Hwang et al., 2000; Shin et al., 2000). Bradykinin, a potent inflammatory mediator, is known to activate VR1 via protein kinase C (PKC), phosphatidylinositol-4,5-bisphosphate, or lipoxygenase-dependent pathways (Shin et al., 2002; Premkumar et al., 2000; Chuang et al., 2001) suggesting that VR1 has a role in mediating inflammatory pain. Further evidence of the pathophysiological role of VR1 is provided by VR1 knockout experiments in mice (Caterina et al., 2000; Davis et al., 2000), where thermal hyperalgesia induced by inflammation is reduced in the knockout mice, suggesting that VR1 is a key element in the generation of nociceptive signals in sensory neurons.

In order to characterize the VR1 receptor, mutagenesis studies and the use of chimeric receptors have begun to identify molecular regions and residues involved in ligand binding. By using capsaicin as a ligand, it was shown that both the N-terminal and the C-terminal cytoplasmic regions are essential for binding, and specifically the Arg-114 and Glu-761 amino acid residues are critical (Jung et al., 1999; Jung et al., 2002) for capsaicin activation of VR1. Based on work with chimeric receptors it has also been suggested that a region around TM3 is involved in the binding of capsaicin to the receptor (Jordt and Julius, 2002). A 5′-splice variant (VR.5′sv), isolated from rat DRG, has also been described (Schumacher et al., 2000). This splice variant is lacking most of the N-terminal cytosolic domain and the ankyrin repeat elements and is insensitive to capsaicin, suggesting that the cytoplasmic domain is essential for a functioning receptor.

Quantitative PCR has been used to determine the expression level of hVR1 in CNS as well as in peripheral tissues. The highest level of expression was found in dorsal root ganglia (DRG) and a low level of VR1 expression was also observed in several peripheral tissues including kidney, liver and pancreas (Hayes et al., 2000).

### SUMMARY OF THE INVENTION

The invention provides a variant of the Vanilloid Receptor. For convenience, as used herein, the wild-type Vanilloid Receptor is termed “VR1.” The variant of the invention is termed “VR1A” and is further defined herein.

The invention provides isolated polynucleotides encoding VR1A (SEQ ID NO:4) accounting for the degeneracy of the genetic code. An example of the polynucleotides of the invention is SEQ ID NO:2. The invention also comprises polynucleotides having at least 70-99% identity to the sequence of SEQ ID NO:23. Preferably, the polynucleotide sequences comprise a sequence having at least 70-99% identity to the sequence of SEQ ID NO:23 and encoding at least one ankyrin repeat. More preferably, the polynucleotides of the invention comprise a sequence having at least 70-99% identity to the sequence of SEQ ID NO:23, a sequence encoding a plurality of ankyrin repeats and a sequence encoding a plurality of transmembrane-spanning domains. Even more preferably, the polynucleotides of the invention comprise a sequence having at least 70-99% identity to the sequence of SEQ ID NO:23, a sequence encoding a plurality of ankyrin repeats, a sequence encoding a plurality of transmembrane-spanning domains and a sequence encoding a pore-loop. The ankyrin repeats may be any known ankyrin repeat, but are preferably at least one repeat selected from the group consisting of SEQ ID NO:24, SEQ ID NO:25, SEQ ID NO:26. In one embodiment of the invention, the repeats consist of SEQ ID NO:24, SEQ ID NO:25 and SEQ ID NO:26. Preferably, there are a plurality of ankyrin repeats. Even more preferably, there are three ankyrin repeats. The transmembrane spanning domains are encoded by any known sequence for a transmembrane region. In some embodiments of the invention, the transmembrane domains are encoded by a plurality of sequences selected from the group consisting of SEQ ID NO:27, SEQ ID NO:43, SEQ ID NO:28, SEQ ID NO:44, SEQ ID NO:29; SEQ ID NO:45, SEQ ID NO:30, SEQ ID NO:46, SEQ ID NO:49, SEQ ID NO:31, SEQ ID NO:32, and SEQ ID NO:47. In a particular embodiment, the transmembrane region is encoded by a polynucleotide sequence comprising SEQ ID NO:27 (TM1), SEQ ID NO:28 (TM2), SEQ ID NO:29 (TM3), SEQ ID NO:30 (TM4), SEQ ID NO:31 (TM5), and SEQ ID NO:32 (TM6). The pore-loop may be encoded by any known sequence for a pore-loop. In a particular embodiment, the pore-loop is encoded by the sequence of SEQ ID NO:51 or SEQ ID NO:52. The invention further comprises polynucleotides that are complementary to these sequences.

The invention also provides polypeptides of VR1A. The polypeptides comprise at least 15-50 contiguous amino acids of SEQ ID NO:20. In one embodiment, the polypeptide of the invention comprises at least 15-50 contiguous amino acids of SEQ ID NO:20 and at least one ankyrin domain. In other embodiments, the polypeptides of the invention comprise a plurality of ankyrin repeats. Although the ankyrin repeats may be any ankyrin repeats known, in some embodiments the ankyrin repeats are selected from the group consisting of those having the amino acid sequence of SEQ ID NO:10, SEQ ID NO:11, SEQ ID NO:12, SEQ ID NO:22, SEQ ID NO:33; and SEQ ID NO:34. In further embodiments of the invention, the polypeptide comprises at least 15-50 contiguous amino acids of SEQ ID NO:20, a plurality of ankyrin repeats and a plurality of transmembrane-spanning domains. Although any known membrane-spanning domain may be included, in some embodiments, the transmembrane domains have the amino acid sequences selected from the group consisting of SEQ ID NO:13, SEQ ID NO:14, SEQ ID NO:15; SEQ ID NO:16, SEQ ID NO:17, SEQ ID NO:18, SEQ ID NO:38, SEQ ID NO:39, SEQ ID NO:40, SEQ ID NO:41, SEQ ID NO:42 and SEQ ID NO:48. Although any sequence of a pore-loop may be included in the polypeptide of the invention, in particular embodiments, the pore-loop has the amino acid sequence of SEQ ID NO:19 or SEQ ID NO:50. In a particular embodiment of the invention, the polypeptide comprises at least 25-50 contiguous amino acids of SEQ ID NO:20, and SEQ ID NOs:10-19. In some embodiments of the invention the he polypeptides have an amino acid sequence that is 80-99% identical to SEQ ID NO:4. In some embodiments, the polypeptide has the amino acid sequence of SEQ ID NO:4. The invention includes polypeptides having conservative amino acid substitutions that do not substantially affect the biological activity of VR1A. In other embodiments, the invention includes polypeptides having non-conservative amino acid substitutions that modulate the biological activity of VR1A. The invention specifically provides nucleic acid sequences that encode any of these polypeptides.

The invention also provides expression vectors for expressing the polypeptides of the invention. The vectors may have one or more control elements enabling efficient expression of the polypeptides. The expression vectors comprise the nucleic acid molecules described herein for VR1A. The control elements may be provided to enable regulated or tissue-specific expression of the polypeptides of the invention. The invention includes methods for expressing a recombinant VR1A in a cell containing an expression vector comprising a VR1A nucleotide sequence comprising culturing the cell in an appropriate cell culture medium under conditions that provide for expression of recombinant VR1A by the cell. Optionally, the method may include the step of purifying recombinant VR1A.

The invention further provides antibodies that specifically bind to a polypeptide of the invention. The antibodies may be polyclonal or monoclonal and include human or humanized versions of the antibodies. The antibodies of the invention may also be adapted for use in non-human species, similar to the “humanization” process, but which make antibodies that more closely resemble those of the host species. A non-limiting example of such antibodies are “primatized” antibodies for use in non-human primates. The antibodies also include functional antibody fragments that retain the binding characteristics of the antibody. Antibody fragments may include Fab, Fab′, F(ab′)₂, F(v) fragments and single chain antibodies. In certain embodiments of the invention, the antibodies recognize an epitope involving at least a portion of the N-terminus of VR1A. In other embodiments, the antibodies recognize an epitope within the N-terminus (SEQ ID NO:20) of VR1A.

The invention also provides host cells that express the polypeptides of the invention, as well as cells expressing the antibodies and antibody fragments of the invention. The host cells may be immortal cell lines. The invention also provides a method of producing VR1A by culturing the host cells under conditions that permit expression of the VR1A. VR1A may be further purified from cell cultures or in association with cell membranes.

The invention also provides compositions comprising a VR1A nucleic acid of the invention, an expression vector of the invention, a polypeptide of the invention or an antibody of the invention in combination with a pharmaceutically acceptable carrier. The invention also provides methods of treating an individual with a VR1A associated disease or disorder comprising administering a composition of the invention.

The invention also provides methods for identifying modulatory compounds, such as agonists and antagonists of VR1A. The method comprises (a) providing a polypeptide comprising the amino acid sequence SEQ ID NO:4, or a fragment thereof which substantially retains the activity of said polypeptide; (b) contacting a candidate compound with said polypeptide or fragment; (c) measuring the effect of said candidate compound on the activity of said polypeptide or fragment; and, for antagonists, (d) selecting a compound which shows at least a 50% increased or decreased effect on the level or duration of said activity. For agonists step (d) would include selecting a compound which shows at least a 50% increase on the level or duration of said activity. For antagonists step (d) would include selecting a compound which shows at least a 50% decrease on the level or duration of said activity.

The activities of VR1A that can be assessed in this regard include, capsaicin binding, heat sensitivity, pH sensitivity, resiniferatoxin stimulation, capsazepine inhibitory activity, and Ca²⁺ influx.

The invention further provides for identifying an individual having a disease or disorder involving cells which comprise a VR1A encoding sequence transcript by known forms of genetic screening, including hybridization analysis and PCR analysis. An individual may also be identified using a protein-based analysis using antibodies that specifically bind to the VR1A.

For example, the invention also provides a method for identifying an individual having a disease or disorder involving cells which comprise a VR1A encoding sequence transcript, comprising the steps of: (a) obtaining a biological sample from said individual to be tested; (b) providing a probe comprising at least one of the nucleic acid molecules of claim 1; (c) contacting said biological sample with said probe under conditions allowing hybridization of said probe with said transcript to form detectable probe-transcript hybridization complexes; and (d) detecting probe-transcript hybridization complexes and comparing the level of said complexes to the level of probe-transcript hybridization complexes detected in a biological sample obtained from a healthy individual, wherein a deviation from the level of the hybridization complexes in said healthy individual indicates a high probability that the tested individual from which the sample was obtained has one of the diseases or disorders involving cells which comprise the VR1A encoding sequence transcript. The invention also provides a method for identifying an individual having a disease or disorder involving cells which comprise a VR1A encoding sequence transcript, comprising the steps of: (a) obtaining a biological sample from said individual to be tested containing mRNA; (b) providing oligonucleotide primers comprising at least one polynucleotide comprising at least 15 consecutive nucleotides of SEQ ID NO:23 and at least one polynucleotide comprising at least 15 consecutive nucleotides of SEQ ID NO:2 wherein said polynucleotides are from opposing strands of DNA; (c) contacting said biological sample with said oligonucleotide primers under conditions allowing reverse transcription and amplification of cDNA to form detectable amplified segment of DNA; and (d) detecting said amplified segment of cDNA and comparing the level of said amplified segment of cDNA to the level of amplified segment of cDNA detected in a biological sample obtained from a healthy individual, wherein a deviation from the level of amplified segment of cDNA in said healthy individual indicates a high probability that the tested individual from which the sample was obtained has one of the diseases or disorders involving cells which comprise the VR1A encoding sequence transcript. The oligonucleotide primers may be any length between 12 and 50 nucleotides in length. Preferably, the oligonucleotides are at least 15 nucleotides in length.

The invention also provides a method for identifying an individual having a high probability of having a disease or disorder involving cells which express the VR1A protein, comprising: (a) obtaining a biological sample from said individual to be tested; (b) contacting said sample with the antibody of claim 12 under conditions enabling the formation of a detectable antibody-antigen complex; and (c) detecting said antibody-antigen complex and comparing the level of said complex to the level of antibody-antigen complexes detected in a sample, obtained from a healthy individual, wherein a deviation from the level of antibody-antigen complexes detected in a sample obtained from said healthy individual indicates a high probability that the tested individual has a disease or disorder involving cells which express the VR1A protein. Such diseases and disorders include, for example, but not by way of limitation, pancreatitis.

The invention also provides methods for identifying a peptide that specifically binds to a VR1A polypeptide by (a) incubating cells expressing VR1A with a phage display peptide library under conditions permitting binding of VR1A to displayed peptides; (b) washing the cells to remove unbound phages; (c) eluting bound phage from the cells; (d) incubating the eluted phage with cells expressing the original protein sequence; (e) collecting the phage that does not bind in step (d); (f) amplifying the phage not bound in step (d); and (g) determining the display peptide sequence of the bound phage.

The invention provides a method for the prevention or treatment of a disorder or disease in an individual where a therapeutically beneficial effect may be achieved by inhibiting or preventing the expression of VR1A in cells of said individual, comprising administering to said individual a therapeutically effective amount of a nucleic acid molecule of the invention, a polypeptide of the invention and/or an antibody of the invention in a pharmaceutically acceptable carrier. Such disorder or disease may be, for example, pancreatitis.

The invention also provides a method of identifying an antibody that specifically binds to VR1A comprising: (a) providing a population of antibody molecules or antibody fragments; (b) screening the members of said population for binding to the polypeptide of claim 5; (c) selecting members of the screened population that bind to the polypeptide of claim 5; (d) screening the selected members for binding to the original protein; and (e) choosing the selected members in (c) that do not bind to the original protein in (d).

The invention also provides a method for the prevention or treatment of a disorder or disease in an individual where a therapeutically beneficial effect may be achieved by administration of an agonist or antagonist of VR1A in cells of said individual, comprising administering to said individual a therapeutically effective amount of a VR1A agonist or antagonist. Such disorder or disease may include, for example, pancreatitis, urinary incontinence, inflammatory conditions of the kidney, and inflammatory conditions of the gastrointestinal tract.

### BRIEF DESCRIPTION OF THE DRAWINGS

FIG. 1A-1D shows a DNA sequence comparison of VR1 (SEQ ID NO:1) and VR1A (SEQ ID NO:2). The initiation codons for VR1 and VR1A, respectively, are outlined in bold letters.

FIG. 2A-2B shows an amino acid comparison of the VR1 (SEQ ID NO:3) and VR1A (SEQ ID NO:4) receptor proteins, respectively. The ankyrin domains are underlined, transmembrane domains are shaded and the pore-loop region is double underlined.

FIG. 3 shows QPCR determination of VR1A messenger RNA in various tissues as determined by the ratio of VR1A concentration in relation to the concentration of a housekeeping enzyme.

FIG. 4 shows activation by capsaicin of VR1 and VR1A cell lines stably expressing VR1 and VR1A, respectively as measured by the Ca²⁺ influx assay.

FIG. 5 shows activation by resiniferatoxin of VR1 and VR1A in stable cell lines as measured by the Ca²⁺ influx assay.

FIG. 6 shows inhibition of capsaicin-induced activity of human VR1 and VR1A cell lines, respectively, by capsazepine, as measured by the Ca²⁺ influx assay.

FIG. 7 shows inhibition of resiniferatoxin-induced activation of human VR1 and VR1A cell lines, respectively, by capsazepine, as measured by the Ca²⁺ influx assay.

### DETAILED DESCRIPTION OF ILLUSTRATIVE EMBODIMENTS

The reference works, patents, patent applications, and scientific literature, including accession numbers to GenBank database sequences, that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Various definitions are made throughout this document. Most words have the meaning that would be attributed to those words by one skilled in the art. Words specifically defined either below or elsewhere in this document have the meaning provided in the context of the present invention as a whole and as are typically understood by those skilled in the art. Any conflict between an art-understood definition of a word or phrase and a definition of the word or phrase as specifically taught in this specification shall be resolved in favor of the latter. Headings used herein are for convenience and are not to be construed as limiting.

Standard reference works setting forth the general principles of recombinant DNA technology known to those of skill in the art include Ausubel et al., CURRENT PROTOCOLS IN MOLECULAR BIOLOGY, John Wiley & Sons, New York, 1998; Sambrook et al., MOLECULAR CLONING: A LABORATORY MANUAL, 2D ED., Cold Spring Harbor Laboratory Press, Plainview, N.Y., 1989; Kaufman et al., Eds., HANDBOOK OF MOLECULAR AND CELLULAR METHODS IN BIOLOGY AND MEDICINE, CRC Press, Boca Raton, 1995; McPherson, Ed., DIRECTED MUTAGENESIS: A PRACTICAL APPROACH, IRL Press, Oxford, 1991.

Structure of VR1A Protein

VR1A is a 850 amino acid protein having three putative ankyrin domains, 6 putative transmembrane domains, and one putative pore-loop domain within SEQ ID NO:4. The ankyrin domains span amino acids 201-233 (SEQ ID NO:10), amino acids 248-280 (SEQ ID NO:11), and amino acids 333-365 (SEQ ID NO:12). The transmembrane domains span amino acids 434-455 (SEQ ID NO:13), 480-495 (SEQ ID NO:14), 510-531 (SEQ ID NO:15), 543-569 (SEQ ID NO:16), 577-596 (SEQ ID NO:17) and 656-684 (SEQ ID NO:18). The pore-loop domain spans amino acids 625-646 (SEQ ID NO:19). The amino acids 149-850 of VR1A (SEQ ID NO:4) are identical to amino acids 151-852 of VR1 (SEQ ID NO:3) with the exception of an amino acid change of Arg→Lys at amino acid 332 of VR1 (SEQ ID NO:3)/330 (SEQ ID NO:4), and an amino acid change of Met→Val at amino acid 552 of VR1 (SEQ ID NO:3)/550 (SEQ ID NO:4). The VR1A splice variant (SEQ ID NO:4) has a different N-terminal region as compared to VR1 (SEQ ID NO:3). The N-terminus of VR1A is a unique 148 amino acid region (SEQ ID NO:20), which differs considerably from the N-terminal 150 amino acids of VR1 (SEQ ID NO:21), sharing only 12% identity. The amino acid changes at 332 and 552 of VR1 are not critical, and this substitution may be made without affecting the biological activity of VR1A. The VR1A N-terminus is encoded by SEQ ID NO:23. The VR1 N-terminus is encoded by SEQ ID NO:54.

Ankyrin repeats are tandemly repeated modules of about 33 amino acids. They occur in a large number of functionally diverse proteins mainly from eukaryotes. The conserved fold of the ankyrin repeat unit is known from several crystal and solution structures. Each repeat folds into a helix-loop-helix structure with a beta-hairpin/loop region projecting out from the helices at a 90° angle. The repeats stack together to form an L-shaped structure (Washington University, St. Louis, Pfam database).

The transmembrane domains of VR1A may be any transmembrane region to allow the protein to be situated in the membrane as in the native molecule. Transmembrane domains are generally hydrophobic stretches of amino acids and may be predicted using any of the computer programs that analyze amino acid sequences to generate data on hydrophobicity of protein sequences.

The pore-loop region forms the selectivity filter of Ca²⁺ ion channels (Ellinor et al. (1995) Neuron 15:1121-1132. Pore loop domains are known for many proteins.

In one embodiment, VR1A comprises at least 15 consecutive amino acids of SEQ ID NO:20. In other embodiments of the invention, VR1A comprises at least 20 consecutive amino acids of SEQ ID NO:20. In other embodiments of the invention, VR1A comprises at least 25 consecutive amino acids of SEQ ID NO:20. In other embodiments of the invention, VR1A comprises at least 30 consecutive amino acids of SEQ ID NO:20. In other embodiments of the invention, VR1A comprises at least 35 consecutive amino acids of SEQ ID NO:20. In other embodiments of the invention, VR1A comprises at least 40 consecutive amino acids of SEQ ID NO:20. In other embodiments of the invention, VR1A comprises at least 45 consecutive amino acids of SEQ ID NO:20. In other embodiments of the invention, VR1A comprises at least 50 consecutive amino acids of SEQ ID NO:20.

In some embodiments of the invention, VR1A comprises at least 15, 20, 25, 30, 35, 40, 45, or 50 consecutive amino acids SEQ ID NO:20, and further comprises at least one ankyrin repeat motif. The ankyrin repeat motif may have the amino acid sequence of any known ankyrin repeat. In some embodiments of the invention, for example, the ankyrin repeat is at least one of SEQ ID NO:10, SEQ ID NO: 11, SEQ ID NO:12, SEQ ID NO:22, SEQ ID NO:33 and/or SEQ ID NO:34. The repeat domains may be one, two or, three repeats or more of each of these, or a combination of two, three or four of these or other ankyrin repeats. In a preferred embodiment, the repeat domain is comprised of three ankyrin repeats. In an especially preferred embodiment, the ankyrin repeats are SEQ ID NO:10, SEQ ID NO:11, and SEQ ID NO:12.

In further embodiments of the invention, the polypeptide comprises at least 15, 20, 25, 30, 35, 40, 45, or 50 consecutive amino acids SEQ ID NO:20, a plurality of ankyrin repeats and a plurality of transmembrane-spanning domains. Although any known membrane-spanning domain may be included, in some embodiments, the transmembrane domains have the amino acid sequences selected from the group consisting of SEQ ID NO:13 (TM1), SEQ ID NO:14 (TM2), SEQ ID NO:15 (TM3); SEQ ID NO:16 (TM4), SEQ ID NO:17 (TM5), SEQ ID NO:18 (TM6), or alternative transmembrane regions SEQ ID NO:38 (TM1), SEQ ID NO:39 (TM2), SEQ ID NO:40 (TM3), SEQ ID NO:41 (TM4), SEQ ID NO:42 (TM5) and SEQ ID NO:48 (TM4). Although any sequence of a pore-loop may be included in the polypeptide of the invention, in particular embodiments, the pore-loop has the amino acid sequence of SEQ ID NO:19 or SEQ ID NO:50. In a particular embodiment of the invention, the polypeptide comprises at least 15, 20, 25, 30, 35, 40, 45, or 50 consecutive amino acids SEQ ID NO:20, and SEQ ID NOs:10-19. In some embodiments of the invention the he polypeptides have an amino acid sequence that is 80-99% identical to SEQ ID NO:4. In some embodiments, the polypeptide has the amino acid sequence of SEQ ID NO:4. The invention includes polypeptides having conservative amino acid substitutions that do not substantially affect the biological activity of VR1A. In other embodiments, the invention includes polypeptides having non-conservative amino acid substitutions that modulate the biological activity of VR1A.

Function of VR1A

VR1 and VR1A exhibit various functionalities associated with vanilloid receptors. As shown in the examples herein, despite the sequence divergence at the N-terminus of the two proteins, VR1A functions similarly to VR1 in terms of capsaicin and sensitivity to resiniferatoxin (an agonist) and capsazepine (an inhibitor), as well as having similar Ca²⁺ influx activity. While not wishing to be bound by any particular theory, it is believed that VR1A is important for potentially binding to different natural ligands than VR1. These natural ligands may be present in different tissues in which VR1A is preferentially expressed over VR1.

In some embodiments of the invention, VR1A has substantially the same capsaicin activity as VR1. In other embodiments, VR1A has substantially the same capsazepine activity as VR1. In other embodiments, VR1A has substantially the same Ca²⁺ influx activity as VR1. In other embodiments, VR1A has substantially the same resiniferatoxin activity as VR1. In other embodiments, VR1A has substantially the same pH sensitivity as VR1. In other embodiments, VR1A has substantially the same heat sensitivity as VR1.

Polynucleotides of VR1A

VR1 is encoded by polynucleotides, which, through the degeneracy of the genetic code, may be represented by a myriad of molecules, each encoding the same protein. The nucleic acid molecules of the invention include those that encode a polypeptide of the invention, and include those specifically described above. In some embodiments, the polynucleotides of the invention encode the amino acid sequences set forth in SEQ ID NO:2 or comprising SEQ ID NO:20.

The nucleic acid molecules of the invention include those that encode a full-length VR1A, as well as functional or antigenic fragments thereof. “Functional fragments” of VR1A are those that retain some biological activity of the native VR1A. The structure of VR1A includes functional domains such as the ankyrin repeats, transmembrane spanning domains and a pore-loop. These structural motifs correlate to activities of VR1A activity and loss of such activity or enhancement of such activity may be determined using biological assays as described herein and as are well-known in the art. For convenience, “VR1A” is the designation given to the variant of VR1 that has a unique N-terminal region (SEQ ID NO:20). However, various permutations and derivatives of the full-length VR1A are within the scope of the term VR1A, provided the molecules do not encompass native VR1.

Although the polypeptides of the invention may be encoded by any nucleotide sequence resulting in the subject amino acid sequence, and each of these is contemplated by the invention, in one embodiment, the unique VR1A N-terminus (amino acids shown in SEQ ID NO:20) is encoded by SEQ ID NO:23. Likewise, in specific embodiments the ankyrin repeats of SEQ ID NO:10, SEQ ID NO:11, SEQ ID NO:12, SEQ ID NO:22, SEQ ID NO:33 and SEQ ID NO:34 are encoded by SEQ ID NO:24, SEQ ID NO:25, SEQ ID NO:26, SEQ ID NO:35, SEQ ID NO:36 and SEQ ID NO:37 respectively. In specific embodiments, transmembrane repeats (“TM” with numbering in order of N-terminal to C-terminal in native proteins) of SEQ ID NO:13 (TM1), SEQ ID NO:38 (TM1); SEQ ID NO:14 (TM2), SEQ ID NO:39 (TM2); SEQ ID NO:15 (TM3), SEQ ID NO:40 (TM3); SEQ ID NO:16 (TM4), SEQ ID NO:41 (TM4), SEQ ID NO:48 (TM4); SEQ ID NO:17 (TM5); SEQ ID NO:18 (TM6), SEQ ID NO:42 (TM5) are encoded by SEQ ID NO:27, SEQ ID NO:43, SEQ ID NO:28, SEQ ID NO:44, SEQ ID NO:29; SEQ ID NO:45, SEQ ID NO:30, SEQ ID NO:46, SEQ ID NO:49, SEQ ID NO:31, SEQ ID NO:32, and SEQ D NO:47, respectively. The transmembrane regions have somewhat different sequences as isolates are compared for VR1. Thus substitutions of the VR1A transmembrane regions may be made using these other known sequences. Preferably, a transmembrane domain is substituted with a homologous transmembrane domain (e.g., a TM4 is substituted with a different TM4).

Likewise the pore loop of the VR1A (SEQ ID NO:19 or SEQ ID NO:50) are encoded by SEQ ID NO:52 and SEQ ID NO:51, respectfully. One of these may be selected for incorporation into the VR1A.

An example of a complete VR1A polynucleotide is shown in SEQ ID NO:2.

The polynucleotides may contain mutations that result in amino acid changes that are either conservative or non-conservative. Mutations can be introduced into a nucleic acid sequence of the invention by standard techniques, such as site-directed mutagenesis and PCR-mediated mutagenesis. Conservative amino acid substitutions may be made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (for example, lysine, arginine, and histidine), acidic side chains (for example, aspartic acid, glutamic acid), uncharged polar side chains (for example, glycine, asparagine, glutamine, serine, threonine, tyrosine, cysteine), nonpolar side chains (for example, alanine, valine, leucine, isoleucine, proline, phenylalanine, methionine, tryptophan), beta-branched side chains (for example, threonine, valine, isoleucine), and aromatic side chains (for example, tyrosine, phenylalanine, tryptophan, histidine). Thus, a predicted nonessential amino acid residue is replaced with another amino acid residue from the same side chain family. Alternatively, mutations can be introduced randomly along all or part of a coding sequence, such as by saturation mutagenesis, and the resultant mutants can be screened for biological activity to identify mutants that retain activity. Following mutagenesis, the encoded protein may be expressed by any recombinant technology known in the art and the activity of the protein can be determined.

In some embodiments of the invention the mutations will not significantly alter the biological activity of the protein. “Biological activity” refers to capsaicin binding, heat sensitivity, pH sensitivity, resiniferatoxin activity, capsazepine activity, Ca²⁺ influx, and the like.

The invention also provides oligonucleotide primers for amplifying VR1A. The oligonucleotide primers may be of any length between 12 and 50 nucleotides. Preferably, the primers are at least 15 nucleotides in length. Generally, the primers are selected based on the sequence of SEQ ID NO:23 such that a forward primer anneals to the noncoding strand of DNA and the reverse primer anneals to the coding strand of DNA. The forward primer generally anneals within, or 5′ to the coding region of the N-terminus of VR1A (SEQ ID NO:23). The reverse primer may be anneal anywhere 3′ of the forward primer to allow a detectable amplicon. In some embodiments, the reverse primer anneals to the sequence within SEQ ID NO:23. In other embodiments, the reverse primer anneals to a sequence within VR1A coding sequence, but downstream of the unique N-terminus coding region. In some embodiments, the reverse primer anneals to a section of DNA downstream of the coding region of VR1A. Computer-assisted selection of oligonucleotides may be performed to identify portions of DNA to allow for efficient amplification based on such criteria as maximizing the melting temperature, for example.

The primers may be used in assays to determine whether an individual has a deviation of expression of VR1A in cells as compared to a healthy individual. The PCR assay may be adapted for use as a reverse transcriptase PCR assay as is well-known in the art.

Expression Vectors

Another aspect of the invention pertains to vectors, preferably expression vectors, containing a nucleic acid encoding VR1A polypeptide, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., VR1A polypeptides, mutant forms of VR1A, fusion proteins, etc.).

The recombinant expression vectors of the invention can be designed for expression of VR1A in prokaryotic or eukaryotic cells. For example, VR1A can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc.; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the VR1A expression vector is a yeast expression vector. Examples of vectors for expression in yeast Saccharomyces cerevisiae include pYepSec1 (Baldari, et al., (1987) EMBO J 6:229-234), pMFa (Kujan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, VR1A can be expressed in insect cells using baculovirus expression vectors. Baculovirus vectors available for expression of proteins in cultured insect cells (e.g., Spodoptera frugiperda SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells, see, e.g., Chapters 16 and 17 of Sambrook et al., MOLECULAR CLONING: A LABORATORY MANUAL 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to VR1A mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Host Cells

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, VR1A polypeptide can be expressed in bacterial cells such as E. Coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding VR1A or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) VR1A polypeptide. Accordingly, the invention further provides methods for producing VR1A polypeptide using the host cells of the invention. In one embodiment, the method comprises culturing the host cell of invention (into which a recombinant expression vector encoding VR1A has been introduced) in a suitable medium such that VR1A polypeptide is produced. In another embodiment, the method further comprises isolating VR1A from the medium or the host cell.

Transgenic Animals

The host cells of the invention can also be used to produce nonhuman transgenic animals. For example, in one embodiment, a host cell of the invention is a fertilized oocyte or an embryonic stem cell into which VR1A-encoding sequences have been introduced. Such host cells can then be used to create non-human transgenic animals in which exogenous VR1A sequences have been introduced into their genome or homologous recombinant animals in which endogenous VR1A sequences have been altered. Such animals are useful for studying the function and/or activity of VR1A and for identifying and/or evaluating modulators of VR1A activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous VR1A gene has been altered by homologous recombination between the endogenous gene and an exogenous DNA molecule introduced into a cell of the animal, e.g., an embryonic cell of the animal, prior to development of the animal.

A transgenic animal of the invention can be created by introducing VR1A-encoding nucleic acid into the male pronuclei of a fertilized oocyte, e.g., by microinjection, retroviral infection, and allowing the oocyte to develop in a pseudopregnant female foster animal. The human VR1A cDNA sequence of SEQ ID NO:2 or an artificial construct comprising the N-terminus of VR1A, three ankyrin repeats, six transmembrane domains and a pore-loop domain, can be introduced as a transgene into the genome of a non-human animal. In some embodiments, variants of the VR1A are introduced in which the VR1A lacks one or more domains, or has substitutions of wild-type sequences with homologs. Alternatively, a nonhuman homologue of the human VR1A gene, such as a mouse VR1A gene, can be isolated based on hybridization to the human VR1A cDNA (described further above) and used as a transgene. Intronic sequences and polyadenylation signals can also be included in the transgene to increase the efficiency of expression of the transgene. A tissue-specific regulatory sequence(s) can be operably linked to the VR1A transgene to direct expression of VR1A polypeptide to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the VR1A transgene in its genome and/or expression of VR1A mRNA in tissues or cells of the animals. A transgenic founder animal can then be used to breed additional animals carrying the transgene. Moreover, transgenic animals carrying a transgene encoding VR1A can further be bred to other transgenic animals carrying other transgenes.

To create a homologous recombinant animal, a vector is prepared which contains at least a portion of a VR1A gene into which a deletion, addition or substitution has been introduced to thereby alter, e.g., functionally disrupt, the VR1A gene. The VR1A gene can be a human gene (e.g., the cDNA of SEQ ID NO:2), but more preferably, is a non-human homologue of a human VR1A gene. For example, a mouse homologue of human VR1A gene can be used to construct a homologous recombination vector suitable for altering an endogenous VR1A gene in the mouse genome. In one embodiment, the vector is designed such that, upon homologous recombination, the endogenous VR1A gene is functionally disrupted (i.e., no longer encodes a functional protein; also referred to as a “knock out” vector).

Alternatively, the vector can be designed such that, upon homologous recombination, the endogenous VR1A gene is mutated or otherwise altered but still encodes functional protein (e.g., the upstream regulatory region can be altered to thereby alter the expression of the endogenous VR1A polypeptide). In the homologous recombination vector, the altered portion of the VR1A gene is flanked at its 5′ and 3′ ends by additional nucleic acid of the VR1A gene to allow for homologous recombination to occur between the exogenous VR1A gene carried by the vector and an endogenous VR1A gene in an embryonic stem cell. The additional flanking VR1A nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for a description of homologous recombination vectors. The vector is introduced into an embryonic stem cell line (e.g., by electroporation) and cells in which the introduced VR1A gene has homologously recombined with the endogenous VR1A gene are selected (see e.g., Li et al. (1992) Cell 69:915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr. Opin. Biotechnol. 2:823-829; PCT International Publication Nos.: WO 90/11354; WO 91/01140; WO 92/0968; and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Assays

The invention provides methods for determining whether a compound can specifically bind to VR1A and methods for determining whether a compound can modulate VR1A as either an agonist or an antagonist. Agonist and antagonist compounds are useful as analgesics. While not wishing to be bound by any particular theory of operation, it is believed that prolonged receptor desensitization that can occur following exposure to agonists. Agonists, antagonists and inverse agonists are all useful as analgesics, as well as for the prevention and treatment of other conditions, such as treatment of urinary incontinence, prevention of urinary bladder hyper-reflexia and treatment of certain neuropathic pain states such as post herpetic neuralgia, diabetic neuropathy, carpal tunnel syndrome and phantom limb pain in amputees.

VR1A-specific agonists and antagonists have an advantage compared to VR1 agonists with respect to treating inflammatory conditions in peripheral tissues. The VR1A expression levels in peripheral tissues facilitate the development of potent and efficacious agonists and antagonists, while at the same time limit CNS-mediated side effect due to the low level of expression in CNS tissue. However, VR1A agonists or antagonists may also be useful in CNS tissue despite lower levels of VR1A in those tissues as compared with VR1 levels. Inflammatory conditions that may be treated with VR1A agonists or antagonists include, but are not limited to pancreatitis, urinary incontinence, as well as inflammatory conditions present in kidney, GI-tract or other visceral organs innervated by neurons expressing VR1A. The invention thus provides assays for identifying compounds useful (a) as analgesics, (b) for the treatment of urinary incontinence, (c) for the prevention of urinary bladder hyper-reflexia and (d) for the treatment of neuropathic pain states (e.g., post herpetic neuralgia, diabetic neuropathy, carpal tunnel syndrome or phantom limb pain).

The invention in particular provides an assay for determining if a compound binds specifically to VR1A. This assay comprises contacting an experimental sample of either recombinant cells of the invention or isolated membrane preparation of such cells with a labeled capsaicin agonist and a test compound. A second control sample of either recombinant cells expressing the human VR1A or an isolated membrane preparation of such cells is contacted only with labeled capsaicin agonist. The unbound labeled agonist is removed from both samples and the amount of bound label in both the experimental sample and the control sample is determined. The amount of bound label in the experimental sample is compared to the amount of bound label in the control sample. If the experimental sample exhibits a 2-fold decrease, or more preferably a 5-fold decrease or most preferably a 10-fold decrease in the amount of bound labeled capsaicin agonist, the compound in the experimental sample is identified as binding specifically to VR1A.

In the above-described binding assay the labeled capsaicin agonist may be any agonist that is known to bind specifically to VR1A, such as capsaicin or resiniferatoxin and may be labeled by any detectable label. Detectable labels include, but are not limited to, radiolabels, fluorescent labels and colorimetric labels. A particularly preferred labeled capsaicin agonist is [³H]resiniferatoxin. Removal of unbound label may be accomplished by filtering or washing the samples but is not limited to these methods.

The invention also provides functional assays for identifying compounds that act as modulators of VR1A. Such assays can be used to classify compounds as agonists or antagonists of the capsaicin receptor.

This invention provides a method for determining whether a compound is a human VR1A agonist, which comprises contacting a recombinant cell of the invention with the compound under conditions that permit activation of a functional human VR1A response, detecting a functional increase in human VR1A activity, and thereby determining whether the compound is a human VR1A agonist.

In one such embodiment the invention provides an assay for determining if a compound is an agonist of VR1A where the functional response is a change in the concentration of intracellular Ca²⁺. This assay comprises contacting a sample of recombinant cells expressing the human VR1A with an indicator of intracellular Ca²⁺ concentration to yield indicator-loaded cells. After a sufficient incubation period excess indicator is removed from the cells to yield washed, indicator-loaded cells. A potential agonist compound is added to a sample of the washed, indicator-loaded cells. This sample is the experimental sample; the control sample is comprised of washed, indicator-loaded cells to which no potential agonist compound had been added. The concentrations of intracellular Ca²⁺ experimental and control samples are measured by quantitating a change in the indicator of intracellular Ca²⁺. The concentration of intracellular Ca²⁺ in the experimental cells that have been contacted with a potential agonist compound is compared to the concentration of intracellular Ca²⁺ in the control cells. If the experimental sample exhibits a 1.5-fold increase, or more preferably a 5-fold increase or most preferably a 10-fold increase (or any significant increase) in the concentration of intracellular Ca²⁺ the compound in the experimental sample is identified as a VR1A agonist. As used herein and in the Claims, a significant change (e.g., increase or decrease) is one that is significant to the p ≦0.05 level in any standard parametric test of statistical significance, such as the F-test, or the Student's T-test.

Particularly preferred indicators of intracellular Ca²⁺ concentration are membrane permeable calcium sensitive dyes, e.g., Fluo-3 and Fura-2. These dyes produce a fluorescent signal when bound to Ca²⁺. Removal of excess indicator from the indicator-loaded cells may be accomplished by washing or filtering cells, but is not limited to these methods.

This invention provides a method for determining whether a compound is a human VR1A antagonist, which comprises contacting a cell of the invention with the compound in the presence of a known VR1A agonist, such as capsaicin or resiniferatoxin, under conditions that permit the activation of a functional VR1A response, detecting a decrease in human VR1A activity, and thereby determining whether the compound is a human VR1A antagonist.

In one embodiment, the assay to identify compounds that act as antagonists of VR1A comprises contacting a test sample of recombinant cells expressing the human VR1A with an indicator of intracellular Ca²⁺ concentration and a test compound (preferably the cells are pre-loaded with the indicator). A second control sample of recombinant cells expressing the human VR1A is contacted only with the indicator of intracellular Ca²⁺ concentration. After a sufficient incubation period excess indicator of intracellular Ca²⁺ is removed from the test and control cells to yield washed, indicator-loaded test and control cells. An agonist of the VR1A is added to the washed, indicator-loaded cells to yield agonist-contacted test cells and agonist-contacted control cells. The concentration of intracellular Ca²⁺ in the agonist-contacted test cells and the agonist-contacted control cells is measured by measuring changes in the properties of the indicator of intracellular Ca²⁺ concentration. The concentration of intracellular Ca²⁺ in the agonist-contacted test cells is compared to that in agonist-contacted control cells. A test compound for which this comparison indicates that the concentration of intracellular Ca²⁺ in the agonist-contacted test cells is significantly less, to the p ≦0.05 level, than the concentration of intracellular Ca²⁺ in the agonist-contacted control cells is identified as an antagonist of VR1A.

As in the assay for agonists of the VR1A, particularly preferred indicators of intracellular Ca²⁺ concentration are the membrane permeable calcium sensitive dyes, Fluo-3 and Fura-2. These dyes produce a fluorescent signal when bound to Ca²⁺. Removal of excess indicator from the indicator-loaded cells may be accomplished by any suitable method, such as washing or filtering cells.

VR1A appears to be more abundant in peripheral tissues rather than in nervous tissue. The highest expression of VR1A found in tissue expression studies was in pancreas, however, VR1A is also found in brain, testis, and dorsal root ganglia.

Pharmaceutical Compositions

The VRA1 nucleic acid molecules, VRA1 polypeptides, and anti-VRA1 antibodies (also referred to herein as “active ingredients”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a VRA1 polypeptide or anti-VRA1 antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Antibodies that Specifically Recognize VR1A

The invention provides antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies), including compounds which include CDR sequences which specifically recognize VR1A, or fragments of VR1A wherein the epitope comprises at least a portion of the amino acid sequence of SEQ ID NO:20.

Antibody fragments, including Fab, Fab′, F(ab′)₂, and Fv, are also provided by the invention. The term “specific for,” when used to describe antibodies of the invention, indicates that the variable regions of the antibodies of the invention recognize and bind VR1A exclusively (i.e., are able to distinguish VR1A from other polypeptides by virtue of measurable differences in binding affinity, despite the possible existence of localized sequence identity, homology, or similarity between VR1A and such polypeptides).

It will be understood that specific antibodies may also interact with other proteins (for example, Staphylococcus aureus protein A or other antibodies in ELISA techniques) through interactions with sequences outside the variable region of the antibodies, and, in particular, in the constant region of the molecule. Screening assays to determine binding specificity of an antibody of the invention are well known and routinely practiced in the art. For a comprehensive discussion of such assays, see Harlow et al. (Eds.), ANTIBODIES: A LABORATORY MANUAL; Cold Spring Harbor Laboratory; Cold Spring Harbor, N.Y., 1988, Chapter 6. Antibodies that recognize and bind fragments of VR1A are also contemplated, provided that the antibodies are specific for VR1A. Antibodies of the invention can be produced using any method well known and routinely practiced in the art.

For the production of polyclonal antibodies, various suitable host animals (e.g., rabbit, goat, mouse or other mammal) may be immunized by injection with the native polypeptide, or a synthetic variant thereof, or a derivative of the foregoing. An appropriate immunogenic preparation can contain, for example, recombinantly expressed VR1A polypeptide or a chemically synthesized VR1A polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille calmette-guerin and Corynebacterium parvum, or similar immunostimulatory agents. If desired, the antibody molecules directed against VR1A can be isolated from the mammal (e.g., from the blood) and further purified by well known techniques, such as protein A chromatography to obtain the IgG fraction.

The term “monoclonal antibody” or “monoclonal antibody composition,” as used herein, refers to a population of antibody molecules that contain only one species of an antigen binding site capable of immunoreacting with a particular epitope of VR1A. A monoclonal antibody composition thus typically displays a single binding affinity for a particular VR1A polypeptide with which it immunoreacts. For preparation of monoclonal antibodies directed towards a particular BLAA polypeptide, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., (1983) Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al. (1983) Proc Natl Acad Sci USA 80:2026-2030) or by transforming human B-cells with Epstein Barr Virus in vitro (see Cole, et al. (1985) In: MONOCLONAL ANTIBODIES AND CANCER THERAPY, Alan R. Liss, Inc., pp. 77-96). Each of the above citations are incorporated herein by reference in their entirety.

According to the invention, techniques can be adapted for the production of single-chain antibodies specific to a VR1A polypeptide (see e.g., U.S. Pat. No. 4,946,778). In addition, methodologies can be adapted for the construction of Fab expression libraries (see e.g., Huse, et al., 1989 Science 246: 1275-1281) to allow rapid and effective identification of monoclonal Fab fragments with the desired specificity for a BLAA polypeptide or derivatives, fragments, analogs or homologs thereof. Non-human antibodies can be “humanized” by techniques well known in the art. See e.g., U.S. Pat. No. 5,225,539. Antibody fragments that contain the idiotypes to a BLAA polypeptide may be produced by techniques known in the art including, but not limited to: (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing agent and (iv) Fv fragments.

Additionally, recombinant anti-VR1A antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PTC/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better et al. (1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst 80:1553-1559); Morrison (1985) Science 229:1202-1207; Oi et al. (1986) BioTechniques 4:214; Jones et al. (1986) Nature 321:552-525; Verhoeyan et al. (1988) Science 239:1534; and Beidler et al (1988) J Immunol 141:4053-4060. Each of the above citations are incorporated herein by reference in their entirety.

In one embodiment, methodologies for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a VR1A polypeptide is facilitated by generation of hybridomas that bind to the fragment of a VR1A polypeptide possessing such a domain. Antibodies that are specific for an Ig-like domain within a VR1A polypeptide, or derivatives, fragments, analogs or homologs thereof, are also provided herein.

Anti-VR1A antibodies may be used in methods known within the art relating to the localization and/or quantitation of a VR1A polypeptide (e.g., for use in measuring levels of the VR1A polypeptide within appropriate physiological samples, for use in diagnostic methods, for use in imaging the polypeptide, and the like). In a given embodiment, antibodies for VR1A polypeptides, or derivatives, fragments, analogs or homologs thereof, that contain the antibody derived binding domain, are utilized as pharmacologically-active compounds (hereinafter “therapeutics”).

An anti-VR1A antibody (e.g., monoclonal antibody) can be used to isolate VR1A by standard techniques, such as affinity chromatography or immunoprecipitation. An anti-VR1A antibody can facilitate the purification of natural BLAA from cells and of recombinantly produced VR1A expressed in host cells. Moreover, an anti-BLAA antibody can be used to detect VR1A polypeptide (e.g., in a cellular lysate or cell supernatant) in order to evaluate the abundance and pattern of expression of the VR1A polypeptide. Anti-VR1A antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include Iodine-125, Iodine-131, Sulfur-35 or tritium. In addition, the antibodies of the present invention may be conjugated to toxins such as radioisotopes, protein toxins and chemical toxins. Such toxins include, but are not limited to Lead-212, Bismuth-212, Astatine-211, Iodine-131, Scandium-47, Rhenium-186, Rhenium-188, Yttrium-90, Iodine-123, Iodine-125, Bromine-77, Indium-111, Boron-10, Actinide, ricin, adriamycin, calicheamicin, and 5-fluorouracil.

The following examples are merely illustrative of the invention and are not to be construed as limiting the invention in any way.

### EXAMPLES

1. Methods. Basic molecular biology techniques such as plasmid DNA isolation, transformation of E. coli, agarose gel electrophoresis and DNA sequencing, are well known to those skilled in the art and are described in the literature (Ausubel F. M. et al., 1987).

A. Isolation of human VR1 and VR-1A cDNAs: Oligo dT-primed first strand cDNA was made from total RNA isolated from human dorsal root ganglia (BD Bioscience) using Superscript III reverse transcriptase (Life Technologies) and following the manufacturer's recommendation. A RACE ready cDNA (Ambion) from human pancreas was also analyzed. Aliquots of the cDNAs were used in PCR (polymerase chain reaction) experiments using Platinum High Fidelity enzyme (Life Technologies) according to the manufacturer's recommendation. The PCR cycle began at 94° C. for 2 min, followed by 30 cycles of 94° C. for 30 s, 58° C. for 30 s, 68° C. for 3.5 min and a final elongation step at 68° C. for 10 min. The two primers were designed to amplify the entire coding region and were based on the DNA sequence of the hVR1 sequence (Gene Bank accession NM_{—}080706). The primer sequences were CAGAGGATCCAGCAAGGATGAAG (forward primer) (SEQ ID NO:5) and AAGGCCCAGTGTTGACAGTGCTGT (reverse primer) (SEQ ID NO:6). PCR products were purified from agarose gels (Qiagene) and subcloned into the pcDNA3.1-TOPO vector (Invitrogen). DNA was isolated from potential VR1A subclones and analyzed by restriction mapping and DNA sequencing.

B. Generation of a stable cell line: The VR1A construct was transfected into 80-90% confluent cultures of CHO cells grown in F12 media containing 10% fetal bovine serum (Hyclone Laboratories). After 48 hours, cells were transferred to selection medium (growth medium containing 1 mg/ml of G418) and 15 days later individual G418 resistant foci were isolated and expanded. In order to identify a functional cell line, the expanded cells were plated in a 96 well plate and grown to confluency. On the day of testing, cells were loaded with the calcium-sensitive dye Fluo-3AM (Molecular Probes, Eugene, Oreg.) and capsaicin-mediated calcium influx measured with a FlexStation (Molecular Devices) as described below. Functional clones were retested, expanded and cryopreserved.

C. Calcium influx assay: The stable cell lines, expressing human DRG VR1 and human pancreas VR1A, were seeded the day before the assay at 40,000 cells/well density in 96 well black clear bottom assay plates (Corning Inc., Corning, N.Y.). On the day of the assay, the medium of cell plates was changed to 0.2 ml of 1× Hanks' balanced salt solution (Invitrogen, Carlsbad, Calif.) containing 20 mM HEPES, pH 7.4, 1.3 mM freshly made probenecid and 3 μM Fluo-3AM (Molecular Devices, Sunnyvale, Calif.). The cell plates were incubated at 37° C. for one hour and left at room temperature for 30 minutes. Serial dilutions of agonist compounds, starting at 100 μM, were set up in 96 well plates and subsequently used for the analysis. Both plates were loaded to the FlexStation in order to run the assay. The excitation wavelength was 485 nM and the emission wavelength was 583 nM.

2. Results

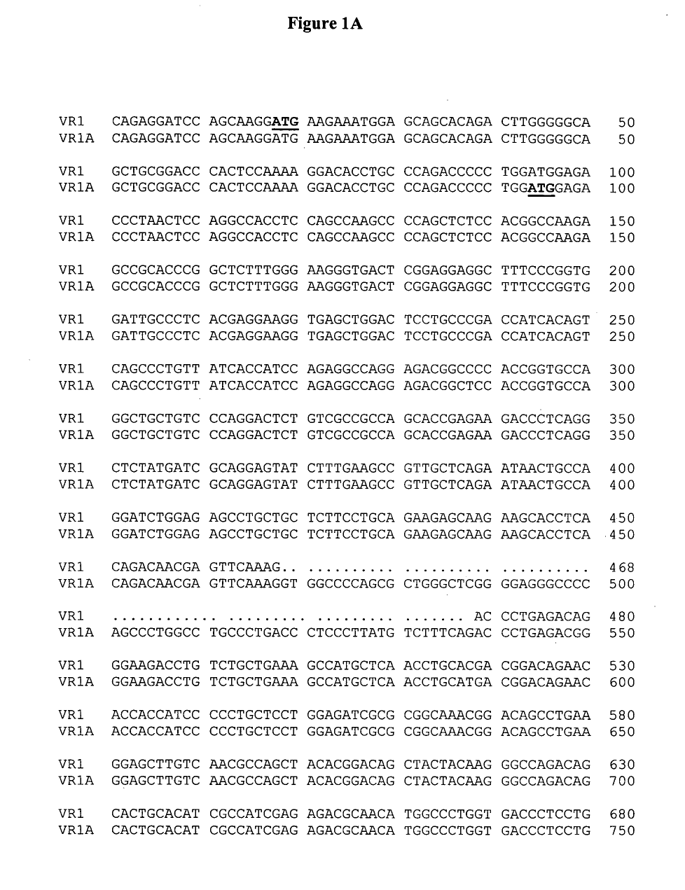

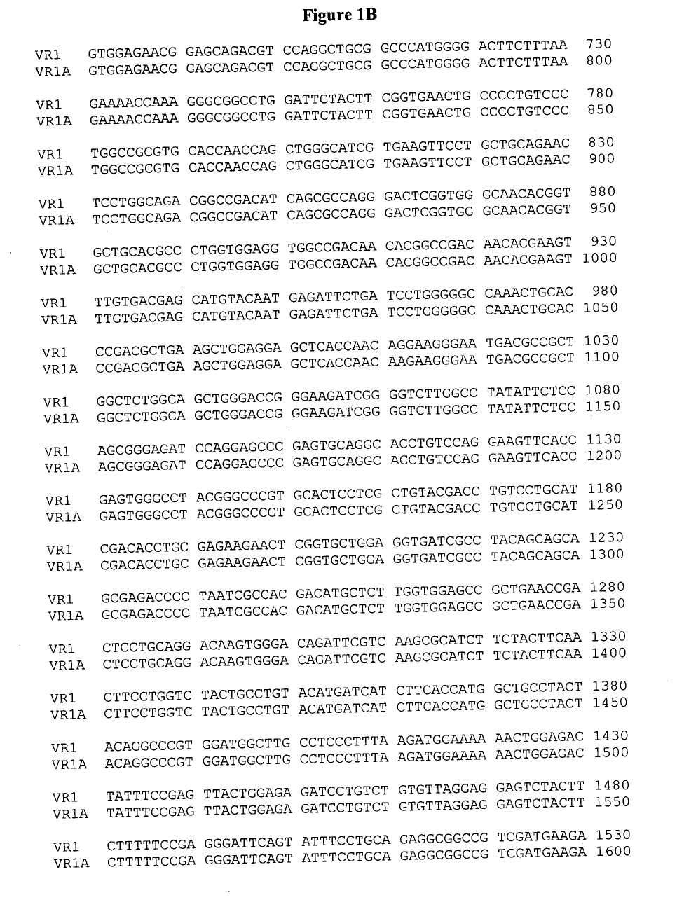

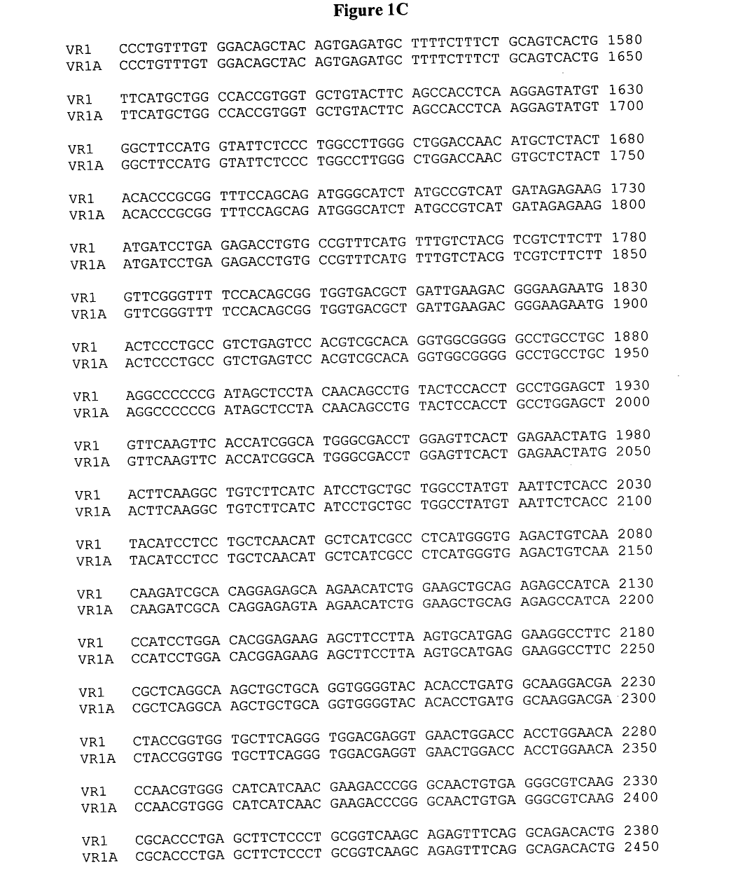

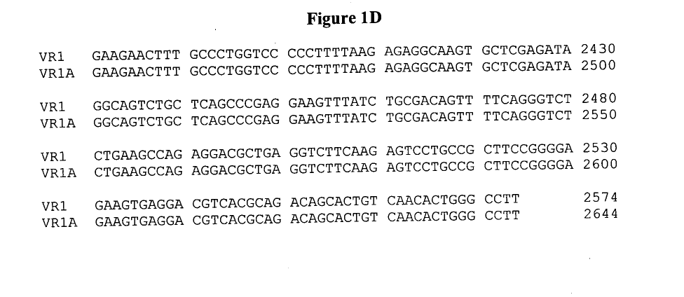

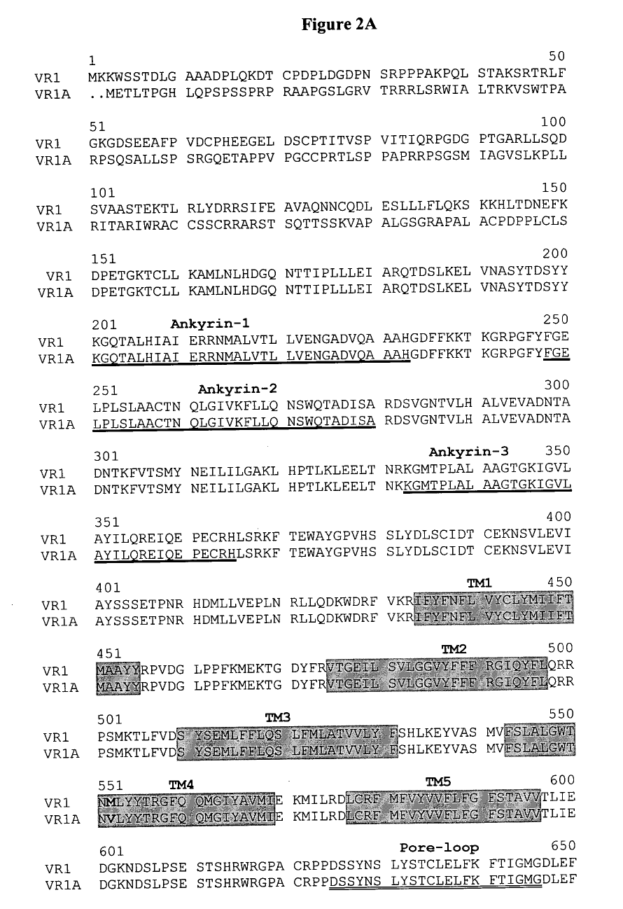

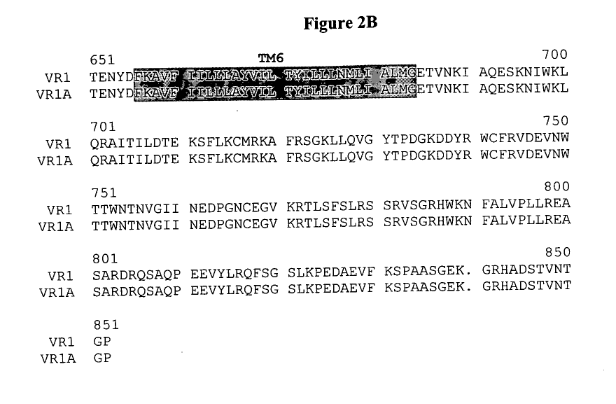

A. Cloning of VR1 and VR1A: PCR experiments with cDNAs from human pancreas and human DRG were conducted as described in the Methods section and DNA sequencing was used to identify VR1 subclones from DRG and VR1A subclones from pancreas. The DNA sequencing results showed that, compared to VR1, VR1A has a 70 bp insertion at position 450 of the VR1 coding sequence (position 468 in FIG. 1). The insertion alters the reading frame and creates a VR1-like protein with 148 unique amino acids at the N-terminal end (FIG. 2) As can be seen in FIGS. 1 and 2, compared to the VR1 coding sequence, the VR1A initiation codon starts at position 94 of the VR1 polynucleotide and the reading frame of the VR1A molecule is identical to VR1 in the polynucleotide located 3′ of the 70 bp insertion. With respect to structural features of VR1A, with the exception of 148 amino acids at the cytosolic N-terminal part, VR1A and VR1 share predicted transmembrane regions, ankyrin repeat domains and potential phosphorylation sites.

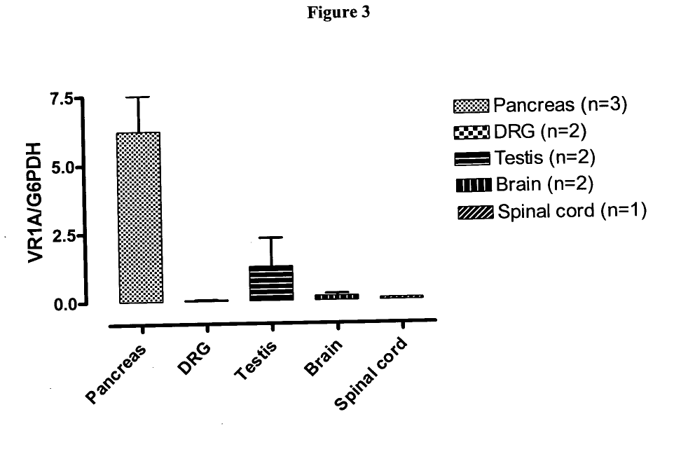

B. VR1A expression: Tissue expression of VR1A was determined using quantitative PCR in a Roche Lightcycler with a SyberGreen indicator as recommended by the manufacturer. The forward primer (5′-GGCCTGCCCTGACCCTCCCTTATGTCTTTC-3′) (SEQ ID NO:7) was selected from the 70 bp insertion sequence and the reverse primer (5′-CTCTCGATGGCGATGTGCAGTGCTGTCTGG-3′) (SEQ ID NO:8) was selected to generate a PCR fragment 400 bp in size. The expression level data (FIG. 3) were normalized to that of a housekeeping enzyme, G6PDH, in order to correct for the quality and amounts of starting material. PCR primers for G6PDH were 5′-TGAGGACCAGATCTACCGCATCGACC-3′ (forward primer) (SEQ ID NO:53) and 5′-ATTCTCCACATAGAGGACGACGGCTGC-3′ (reverse primer) (SEQ ID NO:9). The results show that the highest level of VR1A expression is found in pancreas, but measurable amounts were also found in brain, testis and dorsal root ganglia (DRG). The VR1 receptor is also expressed in these tissues (Hayes et al., 2000), with the highest level observed in DRG. The analysis of VR1 expression conducted by Hayes et al. was performed with PCR primers that do not distinguish between VR1 and VR1A. Still, a comparison between the VR1 results (Hayes et al., 2000) and the VR1A expression data (FIG. 3), suggest that while VR1 is expressed more prominently in the CNS, VR1A is more abundant in peripheral tissues, particularly in pancreatic tissue.

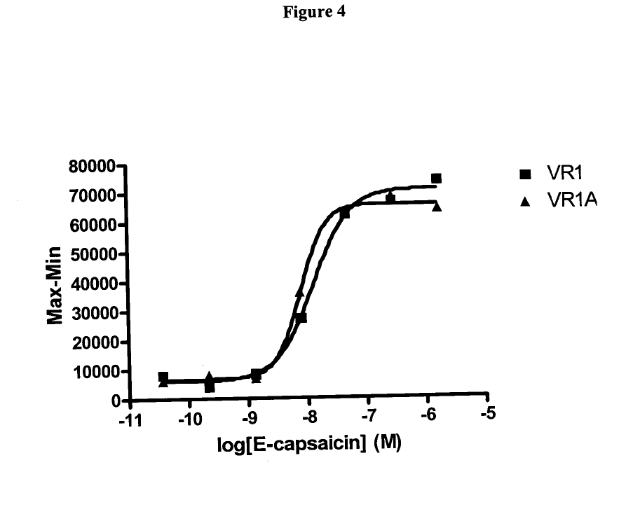

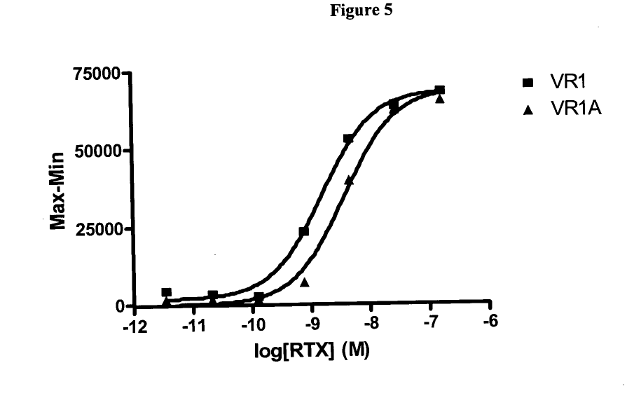

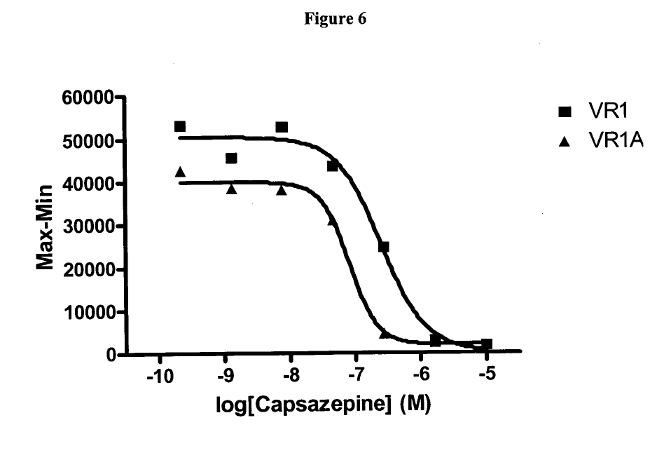

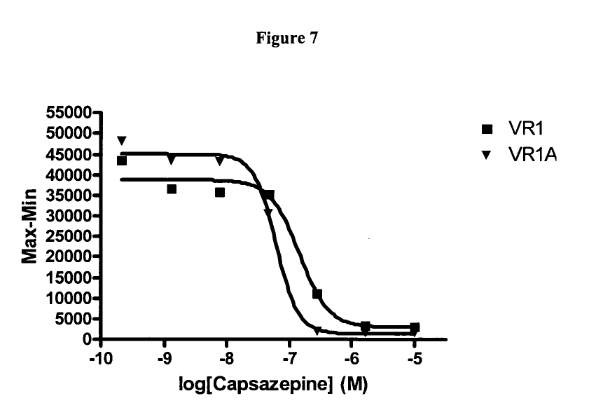

C. VR1A function: In order to determine if the VR1A sequence codes for a functional protein, the VR1A construct was compared to VR1 using the Ca²⁺ influx assay. The comparisons included the use of two VR1 agonists, capsaicin and resiniferatoxin (RTX), and one VR1 antagonist, capsazepine. The determination of EC₅₀ values for the two agonists is shown in FIGS. 4 and 5 and the IC₅₀ values for capsazepine obtained using capsaicin and RTX, respectively, as agonists, are shown in FIGS. 6 and 7. The results are summarized in Table 1. The results show that both agonists activate both VR1A and VR1 receptors, with only minor differences in EC₅₀ values. With respect to the IC₅₀ values for capsazepine, an approximately 5-fold lower value was obtained with RTX as an agonist compared to using capsaicin as an agonist.

TABLE I
Summary of results obtained using the Ca⁺⁺ influx assay
to characterize VR1 and VR1A stable cell lines
expressing the two receptor types, respectively.

| | | IC₅₀ | |
|---|---|---|---|
| | EC₅₀ (nM) | Capsazepine (nM) | N |
| VR1 E-capsaicin | 6.2 ± 0.23 | 194 ± 0.06 | 4 |
| VR1A E-capsaicin | 3.4 ± 0.15 | 60 ± 0.10 | 4 |
| VR1 RTX | 1.6 ± 0.17 | 140 ± 0.80 | 4 |
| VR1A RTX | 5.0 ± 0.08 | 25 ± 0.26 | 4 |

### 3. Conclusions

The cloning and analysis of VR1A isolated from human pancreas total RNA showed that VR1A is a VR1 splice variant generated by the insertion of 70 bp at position 450 of the VR1 coding sequence (position 468 in FIG. 1). VR1A codes for a VR1-like protein, 837 amino acids long, with 148 unique residues at the N-terminal end. The three ankyrin repeat domains located in the cytosolic N-terminal part of VR1 are all maintained in VR1A. The function of VR1A is similar to VR1 as determined by the two VR1 agonists capsaicin and resiniferatoxin (Table I). A rat VR1 splice variant, VR.5′sv (Schumacher et al., 2000), isolated from DRG, was a truncated VR1 receptor lacking two of the ankyrin repeats and was also insensitive to capsaicin. These results suggest that maintaining the three ankyrin repeats in the VR1 splice variant is critical for activation by VR1 agonists.

The expression pattern of VR1A shows that the expression of VR1 and VR1A overlap in both CNS and peripheral tissues. While the highest level of VR1 expression was found in DRG (Hayes et al., 2000, Cortright et al., 2001), VR1A is most abundant in pancreas tissue, raising the possibility that VR1A under certain conditions might have some function unrelated to the function of VR1.

### REFERENCES

Ausubel F. M. et al. (eds), CURRENT PROTOCOLS IN MOLECULAR BIOLOGY, John Wiley & Sons, New York, 1987.
Caterina M J, Schumacher M A, Tominaga M, Rosen T A, Levine J D, Julius D. The capsaicin receptor: a heat-activated ion channel in the pain pathway. Nature 1997; 389: 816-24.
Caterina M J, Leffler A, Malmberg A B, Martin W J, Trafton J, Petersen-Zeitz K R, Koltzenburg M, Basbaum A I, Julius D. Impaired nociception and pain sensation in mice lacking the capsaicin receptor. Science 2000; 288: 241-2.
Caterina M J, Julius D. The vanilloid receptor: a molecular gateway to the pain pathway. Ann. Rev. Neurosci. 2001; 24: 487-517.
Clapham D E. TRP channels as cellular sensors. Nature 2003; 426: 517-24.
Cortright D N, Crandall M, Sanchez J F, Zou T. Krause J E, White G. The tissue distribution and functional characterization of human VR1. Biochem Biophys Res Commun 2001; 281: 1183-9.
Chuang H H, Prescott E D, Kong H, Shields S, Jordt S E, Basbaum A I, Chao M V, Julius D. Bradykinin and nerve growth factor release the capsaicin receptor from Ptdlns(4,5)P2-mediated inhibition. Nature 2001; 411: 957-62.
Davis J B, Gray J, Gunthorpe M J, Hatcher J P, Davey P T, Overend P, Harries M H, Latcham J, Clapham C, Atkinson K, Hughes S A, Rance K, Grau E, Harper A J, Pugh P L, Rogers D C, Bingham S, Randall A, Sheardown S A. Vanilloid receptor-1 is essential for inflammatory thermal hyperalgesia. Nature 2000; 405: 183-7.
Hayes P, Meadows H J, Gunthorpe M J, Harries M H, Duckworth D M, Cairns W, Harrison D C, Clarke C E, Ellington K, Prinjha R K, Barton A J, Medhurst A D, Smith G D, Topp S, Murdock P, Sanger G J, Terrett J, Jenkins O, Benham C D, Randall A D, Gloger I S, Davis J B. Cloning and functional expression of a human orthologue of rat vanilloid receptor-1. Pain 2000; 88: 205-15.
Hwang S W, Cho H, Kwak J, Lee S Y, Kang C J, Jung J, Cho S, Min K H, Suh Y G, Kim D, Oh U. Direct activation of capsaicin receptors by products of lipoxygenases: endogenous capsaicin-like substances. Proc Natl Acad Sci USA 2000; 97: 6155-60.
Jordt S E, Julius D. Molecular basis for species-specific sensitivity to “hot” chili peppers. Cell 2002; 108: 421-30.
Julius D, Basbaum A I. Molecular mechanisms of nociception. Nature 2001; 413: 203-10.
Jung J, Hwang S W, Kwak J, Lee S Y, Kang C J, Kim W B, Kim D, Oh U. Capsaicin binds to the intracellular domain of the capsaicin-activated ion channel. J Neurosci 1999; 19: 529-38.
Jung J, Lee S Y, Hwang S W, Cho H, Shin J, Kang Y S, Kim S, Oh U. Agonist recognition sites in the cytosolic tails of vanilloid receptor 1. J Biol Chem 2002; 277: 44448-54.
Oh U, Hwang S W, and Kim D. Capsaicin activates a nonselective cation channel in cultured neonatal rat dorsal root ganglion neurons. J Neurosci 1996; 16: 1659-67.
Premkumar L S, Ahem G P. Induction of vanilloid receptor channel activity by protein kinase C. Nature 2000; 408: 985-90.
Schumacher M A, Moff I, Sudanagunta S P, Levine J D. Molecular cloning of an N-terminal splice variant of the capsaicin receptor. Loss of N-terminal domain suggests functional divergence among capsaicin receptor subtypes. J Biol Chem 2000; 275: 2756-62.
Shin J, Cho H, Hwang S W, Jung J, Shin C Y, Lee S Y, Kim S H, Lee M G, Choi Y H, Kim J, Haber N A, Reichling D B, Khasar S, Levine J D, Oh U. Bradykinin-12-lipoxygenase-VR1 signaling pathway for inflammatory hyperalgesia. Proc Natl Acad Sci USA 2002; 99: 10150-5.
Szallasi A, Blumberg P M. Vanilloid (Capsaicin) receptors and mechanisms. Pharmacol Rev 1999; 51: 159-212.

What is claimed:

1. An isolated nucleic acid molecule selected from the group consisting of:
(a) a first polynucleotide comprising the sequence of SEQ ID NO:2;
(b) a second polynucleotide comprising a sequence having at least 70-99% identity to the sequence of SEQ ID NO:23;
(c) a third polynucleotide comprising a sequence having at least 70-99% identity to the sequence of SEQ ID NO:23 and encoding a plurality of ankyrin repeats;
(d) a fourth polynucleotide comprising a sequence having at least 70-99% identity to the sequence of SEQ ID NO:23, and a sequence encoding a plurality of ankyrin repeats and a plurality of transmembrane-spanning domains; and
(e) a fifth polynucleotide comprising a sequence having at least 70-99% identity to the sequence of SEQ ID NO:23, a sequence encoding a plurality of ankyrin repeats, a plurality of transmembrane-spanning domains and a pore-loop; and
(f) a sixth polynucleotide comprising the complementary sequence of said first, second, third, fourth or fifth polynucleotide.

2. The polynucleotide of claim 1 wherein said ankyrin repeats comprises a sequence that includes three or more amino acid sequences selected from the group consisting of SEQ ID NO:24, SEQ ID NO:25, SEQ ID NO:26, SEQ ID NO:35, SEQ ID NO:36, and SEQ ID NO:37.

3. The polynucleotide of claim 1 wherein said transmembrane spanning domain comprises a sequence selected from the group consisting of SEQ ID NO:27, SEQ ID NO:43, SEQ ID NO:28, SEQ ID NO:44, SEQ ID NO:29; SEQ ID NO:45, SEQ ID NO:30, SEQ ID NO:46, SEQ ID NO:49, SEQ ID NO:31, SEQ ID NO:32, and SEQ D NO:47.

4. The polynucleotide of claim 1 wherein said pore-loop comprises SEQ ID NO:51 or SEQ ID NO:52.

5. An isolated polypeptide selected from the group consisting of:
(a) a polypeptide having the amino acid sequence of SEQ ID NO:4;
(b) a polypeptide comprising at least 15-50 contiguous amino acids of SEQ ID NO:20 (N-terminal domain of V1A); and
(c) a polypeptide comprising at least 15-50 contiguous amino acids of SEQ ID NO:20 (N-term domain) at least one ankyrin repeat and a plurality of transmembrane-spanning regions; (d) a polypeptide comprising at least 15-50 contiguous amino acids of SEQ ID NO:20 (N-term domain), at least one ankyrin repeat, a plurality of transmembrane-spanning regions and a pore-loop; and
(e) a polypeptide having at least 80-99% identity to SEQ ID NO:4.

6. The polypeptide of claim 5 wherein said ankyrin repeats comprises a sequence selected from the group consisting of SEQ ID NO:10, SEQ ID NO:11, and SEQ ID NO:12.

7. The polypeptide of claim 5 wherein said transmembrane spanning domain comprises a sequence selected from the group consisting of SEQ ID NO:13, SEQ ID NO:14, SEQ ID NO:15; SEQ ID NO:16, SEQ ID NO:17, and SEQ ID NO:18.

8. The polypeptide of claim 5 comprising at least one conservative amino acid substitution which does not substantially affect the biological activity of the polypeptide.

9. The polypeptide of claim 5 comprising at least one non-conservative amino acid substitution which modulates the biological activity of the polypeptide.

10. An antibody selected from the group consisting of:
(a) an antibody which specifically binds to the polypeptide of claim 2; and
(b) fragment of the antibody of (a), wherein said fragment substantially retains the antigen binding characteristics of the antibody of (a).

11. The antibody of claim 10, wherein said antibody is a monoclonal antibody.

12. The antibody of claim 11, wherein the antibody is a human or humanized antibody.

13. The antibody of claim 10, wherein said fragment is selected from the group consisting of a Fab fragment, a Fab′ fragment, a F(ab′)₂ fragment and a F(v) fragment.

14. A method for identifying a candidate modulatory compound of VR1A, comprising:
(a) providing a polypeptide comprising the amino acid sequence SEQ ID NO:4, or a fragment thereof which substantially retains the activity of said polypeptide;
(b) contacting a candidate modulatory compound with said polypeptide or fragment;
(c) measuring the effect of said modulatory compound on the activity of said polypeptide or fragment; and
(d) selecting a compound which shows at least a 50% increased or decreased effect on the level or duration of said activity.

15. The method of claim 14 wherein said modulatory compound is an agonist of VR1A.

16. The method of claim 14 wherein said modulatory compound is an antagonist of VR1A.


SEQUENCE LISTING (54 sequences)

SEQ ID NO: 1 - DNA, 2574 bp, Homo sapiens
cagaggatccagcaaggatgaagaaatggagcagcacagacttgggggcagctgcggacccactccaaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaagaccctgagacagggaagacctgtctgctgaaagccatgctcaacctgcacgacggacagaacaccaccatccccctgctcctggagatcgcgcggcaaacggacagcctgaaggagcttgtcaacgccagctacacggacagctactacaagggccagacagcactgcacatcgccatcgagagacgcaacatggccctggtgaccctcctggtggagaacggagcagacgtccaggctgcggcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgccagggactcggtgggcaacacggtgctgcacgccctggtggaggtggccgacaacacggccgacaacacgaagtttgtgacgagcatgtacaatgagattctgatcctgggggccaaactgcacccgacgctgaagctggaggagctcaccaacaggaagggaatgacgccgctggctctggcagctgggaccgggaagatcggggtcttggcctatattctccagcgggagatccaggagcccgagtgcaggcacctgtccaggaagttcaccgagtgggcctacgggcccgtgcactcctcgctgtacgacctgtcctgcatcgacacctgcgagaagaactcggtgctggaggtgatcgcctacagcagcagcgagacccctaatcgccacgacatgctcttggtggagccgctgaaccgactcctgcaggacaagtgggacagattcgtcaagcgcatcttctacttcaacttcctggtctactgcctgtacatgatcatcttcaccatggctgcctactacaggcccgtggatggcttgcctccctttaagatggaaaaaactggagactatttccgagttactggagagatcctgtctgtgttaggaggagtctacttctttttccgagggattcagtatttcctgcagaggcggccgtcgatgaagaccctgtttgtggacagctacagtgagatgcttttctttctgcagtcactgttcatgctggccaccgtggtgctgtacttcagccacctcaaggagtatgtggcttccatggtattctccctggccttgggctggaccaacatgctctactacacccgcggtttccagcagatgggcatctatgccgtcatgatagagaagatgatcctgagagacctgtgccgtttcatgtttgtctacgtcgtcttcttgttcgggttttccacagcggtggtgacgctgattgaagacgggaagaatgactccctgccgtctgagtccacgtcgcacaggtggcgggggcctgcctgcaggccccccgatagctcctacaacagcctgtactccacctgcctggagctgttcaagttcaccatcggcatgggcgacctggagttcactgagaactatgacttcaaggctgtcttcatcatcctgctgctggcctatgtaattctcacctacatcctcctgctcaacatgctcatcgccctcatgggtgagactgtcaacaagatcgcacaggagagcaagaacatctggaagctgcagagagccatcaccatcctggacacggagaagagcttccttaagtgcatgaggaaggccttccgctcaggcaagctgctgcaggtggggtacacacctgatggcaaggacgactaccggtggtgcttcagggtggacgaggtgaactggaccacctggaacaccaacgtgggcatcatcaacgaagacccgggcaactgtgagggcgtcaagcgcaccctgagcttctccctgcggtcaagcagagtttcaggcagacactggaagaactttgccctggtcccccttttaagagaggcaagtgctcgagataggcagtctgctcagcccgaggaagtttatctgcgacagttttcagggtctctgaagccagaggacgctgaggtcttcaagagtcctgccgcttccggggagaagtgaggacgtcacgcagacagcactgtcaacactgggcctt

SEQ ID NO: 2 - DNA, 2697 bp, Homo sapiens
acagaggatccagcaaggatgaagaaatggagcagcacagacttgggggcaagctgcggacccactccaaaaggacacctgcccagaccccctggatggagaaccctaactccaggccacctccagccaagccccagctctccacggccaagaagccgcacccggctctttgggaagggtgactcggaggaggctttcccggtgagattgccctcacgaggaaggtgagctggactcctgcccgaccatcacagtacagccctgttatcaccatccagaggccaggagacggctccaccggtgccaaggctgctgtcccaggactctgtcgccgccagcaccgagaagaccctcaggactctatgatcgcaggagtatctttgaagccgttgctcagaataactgccaaggatctggagagcctgctgctcttcctgcagaagagcaagaagcacctcaacagacaacgagttcaaaggtggccccagcgctgggctcggggagggccccaagccctggcctgccctgaccctcccttatgtctttcagaccctgagacggaggaagacctgtctgctgaaagccatgctcaacctgcatgacggacagaacaaccaccatccccctgctcctggagatcgcgcggcaaacggacagcctgaaaggagcttgtcaacgccagctacacggacagctactacaagggccagacagacactgcacatcgccatcgagagacgcaacatggccctggtgaccctcctgagtggagaacggagcagacgtccaggctgcggcccatggggacttctttaaagaaaaccaaagggcggcctggattctacttcggtgaactgcccctgtcccatggccgcgtgcaccaaccagctgggcatcgtgaagttcctgctgcagaacatcctggcagacggccgacatcagcgccagggactcggtgggcaacacggtagctgcacgccctggtggaggtggccgacaacacggccgacaacacgaagtattgtgacgagcatgtacaatgagattctgatcctgggggccaaactgcacaccgacgctgaagctggaggagctcaccaacaagaagggaatgacgccgctaggctctggcagctgggaccgggaagatcggggtcttggcctatattctccaagcgggagatccaggagcccgagtgcaggcacctgtccaggaagttcaccagagtgggcctacgggcccgtgcactcctcgctgtacgacctgtcctgcatacgacacctgcgagaagaactcggtgctggaggtgatcgcctacagcagcaagcgagacccctaatcgccacgacatgctcttggtggagccgctgaaccgaactcctgcaggacaagtgggacagattcgtcaagcgcatcttctacttcaaacttcctggtctactgcctgtacatgatcatcttcaccatggctgcctactaacaggcccgtggatggcttgcctccctttaagatggaaaaaactggagacatatttccgagttactggagagatcctgtctgtgttaggaggagtctacttactttttccgagggattcagtatttcctgcagaggcggccgtcgatgaagaaccctgtttgtggacagctacagtgagatgcttttctttctgcagtcactgattcatgctggccaccgtggtgctgtacttcagccacctcaaggagtatgtaggcttccatggtattctccctggccttgggctggaccaacgtgctctactaacacccgcggtttccagcagatgggcatctatgccgtcatgatagagaagaatgatcctgagagacctgtgccgtttcatgtttgtctacgtcgtcttcttagttcgggttttccacagcggtggtgacgctgattgaagacgggaagaatgaactccctgccgtctgagtccacgtcgcacaggtggcgggggcctgcctgcaaggccccccgatagctcctacaacagcctgtactccacctgcctggagctagttcaagttcaccatcggcatgggcgacctggagttcactgagaactatgaacttcaaggctgtcttcatcatcctgctgctggcctatgtaattctcaccatacatcctcctgctcaacatgctcatcgccctcatgggtgagactgtcaaacaagatcgcacaggagagtaagaacatctggaagctgcagagagccatcaaccatcctggacacggagaagagcttccttaagtgcatgaggaaggccttcacgctcaggcaagctgctgcaggtggggtacacacctgatggcaaggacgaactaccggtggtgcttcagggtggacgaggtgaactggaccacctggaacaaccaacgtgggcatcatcaacgaagacccgggcaactgtgagggcgtcaagacgcaccctgagcttctccctgcggtcaagcagagtttcaggcagacactgagaagaactttgccctggtcccccttttaagagaggcaagtgctcgagataaggcagtctgctcagcccgaggaagtttatctgcgacagttttcagggtctactgaagccagaggacgctgaggtcttcaagagtcctgccgcttccggggaagaagtgaggacgtcacgcagacagcactgtcaacactgggcctt

SEQ ID NO: 3 - protein, 839 aa, Homo sapiens
MKKWSSTDLGAAADPLQKDTCPDPLDGDPNSRPPPAKPQLSTAKSRTRLFGKGDSEEAFPVDCPHEEGELDSCPTITVSPVITIQRPGDGPTGARLLSQDSVAASTEKTLRLYDRRSIFEAVAQNNCQDLESLLLFLQKSKKHLTDNEFKDPETGKTCLLKAMLNLHDGQNTTIPLLLEIARQTDSLKELVNASYTDSYYKGQTALHIAIERRNMALVTLLVENGADVQAAAHGDFFKKTKGRPGFYFGELPLSLAACTNQLGIVKFLLQNSWQTADISARDSVGNTVLHALVEVADNTADNTKFVTSMYNEILILGAKLHPTLKLEELTNRKGMTPLALAAGTGKIGVLAYILQREIQEPECRHLSRKFTEWAYGPVHSSLYDLSCIDTCEKNSVLEVIAYSSSETPNRHDMLLVEPLNRLLQDKWDRFVKRIFYFNFLVYCLYMIIFTMAAYYRPVDGLPPFKMEKTGDYFRVTGEILSVLGGVYFFFRGIQYFLQRRPSMKTLFVDSYSEMLFFLQSLFMLATVVLYFSHLKEYVASMVFSLALGWTNMLYYTRGFQQMGIYAVMIEKMILRDLCRFMFVYVVFLFGFSTAVVTLIEDGKNDSLPSESTSHRWRGPACRPPDSSYNSLYSTCLELFKFTIGMGDLEFTENYDFKAVFIILLLAYVILTYILLLNMLIALMGETVNKIAQESKNIWKLQRAITILDTEKSFLKCMRKAFRSGKLLQVGYTPDGKDDYRWCFRVDEVNWTTWNTNVGIINEDPGNCEGVKRTLSFSLRSSRVSGRHWKNFALVPLLREASARDRQSAQPEEVYLRQFSGSLKPEDAEVFKSPAASGEK

SEQ ID NO: 4 - protein, 837 aa, Homo sapiens [sequence omitted]

SEQ ID NO: 5 - DNA, 23 bp, Artificial
  oligonucleotide primer
cagaggatccagcaaggatgaag

SEQ ID NO: 6 - DNA, 24 bp, Artificial
  oligonucleotide primer
aaggcccagtgttgacagtgctgt

SEQ ID NO: 7 - DNA, 30 bp, Artificial
  oligonucleotide primer
ggcctgccctgaccctcccttatgtctttc

SEQ ID NO: 8 - DNA, 30 bp, Artificial
  Oligonucleotide primer
ctctcgatggcgatgtgcagtgctgtctgg

SEQ ID NO: 9 - DNA, 27 bp, Artificial
  oligonucleotide primer
attctccacatagaggacgacggctgc

SEQ ID NO: 10 - protein, 33 aa, homo sapiens
KGQTALHIAIERRNMALVTLLVENGADVQAAAH

SEQ ID NO: 11 - protein, 32 aa, Homo sapiens
GELPLSLAACTNQLGIVKFLLQNSWQTADISA

SEQ ID NO: 12 - protein, 33 aa, Homo sapiens
KGMTPLALAAGTGKIGVLAYILQREIQEPECRH

SEQ ID NO: 13 - protein, 22 aa, Homo sapiens
IFYFNFLVYCLYMIIFTMAAYY

SEQ ID NO: 14 - protein, 23 aa, Homo sapiens
VTGEILSVLGGVYFFFRGIQYFL

SEQ ID NO: 15 - protein, 22 aa, Homo sapiens
SYSEMLFFLQSLFMLATVVLYF

SEQ ID NO: 16 - protein, 27 aa, Homo sapiens
FSLALGWTNMLYYTRGFQQMGIYAVMI

SEQ ID NO: 17 - protein, 20 aa, Homo sapiens
LCRFMFVYVVFLFGFSTAVV

SEQ ID NO: 18 - protein, 29 aa, Homo sapiens
FKAVFIILLLAYVILTYILLLNMLIALMG

SEQ ID NO: 19 - protein, 22 aa, Homo sapiens
DSSYNSLYSTCLELFKFTIGMG

SEQ ID NO: 20 - protein, 148 aa, Homo sapiens
METLTPGHLQPSPSSPRPRAAPGSLGRVTRRRLSRWIALTRKVSWTPARPSQSALLSPSRGQETAPPVPGCCPRTLSPPAPRRPSGSMIAGVSLKPLLRITARIWRACCSSCRRARSTSQTTSSKVAPALGSGRAPALACPDPPLCLS

SEQ ID NO: 21 - protein, 150 aa, Homo sapiens
MKKWSSTDLGAAADPLQKDTCPDPLDGDPNSRPPPAKPQLSTAKSRTRLFGKGDSEEAFPVDCPHEEGELDSCPTITVSPVITIQRPGDGPTGARLLSQDSVAASTEKTLRLYDRRSIFEAVAQNNCQDLESLLLFLQKSKKHLTDNEFK

SEQ ID NO: 22 - protein, 33 aa, Homo sapiens
KGQTALHIAIERRNMTLVTLLVENGADVQAAAN

SEQ ID NO: 23 - DNA, 637 bp, Homo sapiens
cagaggatccagcaaggatgaagaaatggagcagcacagacttgggggcagctgcggacccactccaaaaggacacctgcccagaccccctggatggagacagaggatccagcaaggatgaagaaatggagcagcacagacttgggggcagctgcggacccactccaaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggctccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaaggtggccccagcgctgggctcggggagggccccagccctggcctgccctgaccctcccttatgtctttca

SEQ ID NO: 24 - DNA, 126 bp, Homo sapiens
aacgccagctacacggacagctactacaagggccagacagcactgcacatcgccatcgagagacgcaacatggccctggtgaccctcctggtggagaacggagcagacgtccaggctgcggcccat

SEQ ID NO: 25 - DNA, 147 bp, Homo sapiens
gcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgcc

SEQ ID NO: 26 - DNA, 99 bp, Homo sapiens
aagggaatgacgccgctggctctggcagctgggaccgggaagatcggggtcttggcctatattctccagcgggagatccaggagcccgagtgcaggcac

SEQ ID NO: 27 - DNA, 72 bp, Homo sapiens
aagcgcatcttctacttcaacttcctggtctactgcctgtacatgatcatcttcaccatggctgcctactac

SEQ ID NO: 28 - DNA, 69 bp, Homo sapiens
gttactggagagatcctgtctgtgttaggaggagtctacttctttttccgagggattcagtatttcctg

SEQ ID NO: 29 - DNA, 66 bp, Homo sapiens
agctacagtgagatgcttttctttctgcagtcactgttcatgctggccaccgtggtgctgtacttc

SEQ ID NO: 30 - DNA, 81 bp, Homo sapiens
ttctccctggccttgggctggaccaacatgctctactacacccgcggtttccagcagatgggcatctatgccgtcatgata

SEQ ID NO: 31 - DNA, 60 bp, Homo sapiens
ctgtgccgtttcatgtttgtctacgtcgtcttcttgttcgggttttccacagcggtggtg

SEQ ID NO: 32 - DNA, 99 bp, Homo sapiens
gagaactatgacttcaaggctgtcttcatcatcctgctgctggcctatgtaattctcacctacatcctcctgctcaacatgctcatcgccctcatgggt

SEQ ID NO: 33 - protein, 32 aa, Rattus rattus
GELPLSLAACTNQLAIVKFLLQNSWQPADISA

SEQ ID NO: 34 - protein, 33 aa, Rattus rattus
KGLTPLALAASSGKIGVLAYILQREIHEPECRH

SEQ ID NO: 35 - DNA, 99 bp, Rattus rattus
aagggccagacagcactgcacattgccattgaacggcggaacatgacgctggtgaccctcttggtggagaatggagcagatgtccaggctgcggctaac

SEQ ID NO: 36 - DNA, 99 bp, Rattus rattus
tttggtgagctgcccctgtccctggctgcgtgcaccaaccagctggccattgtgaagttcctgctgcagaactcctggcagcctgcagacatcagcgcc

SEQ ID NO: 37 - DNA, 99 bp, Rattus rattus
aaggggctcacgccactggctctggctgctagcagtgggaagatcggggtcttggcctacattctccagagggagatccatgaacccgagtgccgacac

SEQ ID NO: 38 - protein, 22 aa, Rattus rattus
IFYFNFFVYCLYMIIFTAAAYY

SEQ ID NO: 39 - protein, 23 aa, Rattus rattus
VTGEILSVSGGVYFFFRGIQYFL

SEQ ID NO: 40 - protein, 22 aa, Rattus rattus
SYSEILFFVQSLFMLVSVVLYF

SEQ ID NO: 41 - protein, 27 aa, Rattus rattus
FSLAMGWTNMLYYTRGFQQMGIYAVMI

SEQ ID NO: 42 - protein, 20 aa, Rattus rattus
LCRFMFVYLVFLFGFSTAVV

SEQ ID NO: 43 - DNA, 66 bp, Rattus rattus
atcttctacttcaacttcttcgtctactgcttgtatatgatcatcttcaccgtggctgcctactat

SEQ ID NO: 44 - DNA, 69 bp, Rattus rattus
gtcaccggagagatcttgtctgtgtcaggaggagtctacttcttcttccgagggattcaatatttcctg

SEQ ID NO: 45 - DNA, 66 bp, Rattus rattus
agctacagtgagatacttttctttgtacagtcgctgttcatgctggtgtctgtggtactgtacttc

SEQ ID NO: 46 - DNA, 81 bp, Rattus rattus
ttctccctggccatgggctggaccaacatgctctactatacccgaggattccagcagatgggcatctatgctgtcatgatt

SEQ ID NO: 47 - DNA, 60 bp, Rattus rattus
ctgtgccggtttatgttcgtctacctcgtgttcttgtttggattttccacagctgtggtg

SEQ ID NO: 48 - protein, 27 aa, Homo sapiens
FSLALGWTNVLYYTRGFQQMGIYAVMI

SEQ ID NO: 49 - DNA, 81 bp, Homo sapiens
ttctccctggccttgggctggaccaacgtgctctactacacccgcggtttccagcagatgggcatctatgccgtcatgata

SEQ ID NO: 50 - protein, 22 aa, Rattus rattus
GNSYNSLYSTCLELFKFTIGMG

SEQ ID NO: 51 - DNA, 66 bp, Rattus rattus
ggtaactcttacaacagcctgtattccacatgtctggagctgttcaagttcaccatcggcatgggc

SEQ ID NO: 52 - DNA, 66 bp, Homo sapiens
gatagctcctacaacagcctgtactccacctgcctggagctgttcaagttcaccatcggcatgggc

SEQ ID NO: 53 - DNA, 26 bp, Artificial
  oligonucleotide primer
tgaggaccagatctaccgcatcgacc

SEQ ID NO: 54 - DNA, 367 bp, Homo sapiens
ccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaa